(19) United States
(12) Patent Application Publication
(10) Pub. No.: US 2023/0404467 A1
(43) Pub. Date: Dec. 21, 2023

(54) HEAD MOUNTED CAMERA AND EYE TRACK SYSTEM FOR ANIMALS

(71) Applicant: The Regents of the University of California, Oakland, CA (US)

(72) Inventor: Vikram Pal Singh, San Diego, CA (US)

(21) Appl. No.: 18/318,381

(22) Filed: May 16, 2023

Related U.S. Application Data
(60) Provisional application No. 63/344,338, filed on May 20, 2022.

(51) Int. Cl. A61B 5/00

(52) U.S. Cl. CPC A61B 5/4064; A61B 2503/40

(57) ABSTRACT

A head mounted camera and eye track system for an animal. The system includes a mount configured to the shape of the head of the animal, a vision assembly configured to attach to the mounting, and data collection circuitry. The vision assembly includes an eye camera and a mirror arranged to be outside of a forward field of view of the animal while being angled to provide an image of an eye of the animal to the eye camera. Circuitry is configured to synchronize the data such that the animal's gaze is synchronized with data of a forward view camera or is configured to provide synchronization information regarding the image data from the eye camera in the case of a virtual reality system.

### PRIORITY CLAIM AND REFERENCE TO RELATED APPLICATION

The application claims priority under 35 U.S.C. § 119 and all applicable statutes and treaties from prior U.S. provisional application Ser. No. 63/344,338 which was filed May 20, 2022.

### STATEMENT OF GOVERNMENT INTEREST

This invention was made with government support under grant numbers R01 NS1188457 and UF1 NS116377 awarded by the National Institutes of Health. The government has certain rights in the invention.

### FIELD

A field of the invention is vision neuroscience using small animals (especially non-human-primates), but also including humans. Another field of the invention is high frame rate image acquisition and analysis systems using artificial neural networks. Additional fields include human virtual reality, augmented reality, and gaming systems.

### BACKGROUND

Most of the Vision Neuroscience that is being conducted in the field has the animal restrained and head-fixed. This is particularly uncomfortable for the animal and does not provide any form of realistic simulation of natural animal behavior. Convention tools to study neuro-visual response of an animal fail to provide insight or understanding of brain activity in a real-world scenario that permits an animal to freely move and make its own decisions, interact with the dynamic environment or stimulus provided in an experiment.

### SUMMARY OF THE INVENTION

A preferred embodiment provides head mounted camera and eye track system for an animal. The system includes a mount configured to the shape of the head of the animal, a vision assembly configured to attach to the mounting, and data collection circuitry. The vision assembly includes an eye camera and a mirror arranged to be outside of a forward field of view of the animal while being angled to provide an image of an eye of the animal to the eye camera.

For vision neuroscience studies, the vision assembly also includes a forward-facing view camera to obtain world view data that can be timed to eye camera data. For a VR application, and preferably for other applications, an illumination source that illuminates the animal's eye is included in the vision assembly. The eye tracking can help drive VR displays. Data collection circuitry includes a microcontroller receiving image data from the eye and, where applicable, the view cameras. A preferred system includes circuitry configured to synchronize the data such that the animal's gaze is synchronized with the view camera data or is configured to provide synchronization information regarding the image data from the eye camera in the case of a virtual reality system.

### BRIEF DESCRIPTION OF THE DRAWINGS

FIG. 1 shows a preferred head mounted camera and eye track system for an animal;

FIGS. 2A-2B respectively show a preferred UNET artificial neural network architecture used for pupil detection from recorded eye video and training pairs of raw and masked images;

FIGS. 3A-3B shows how a preferred eye-tracking system of FIG. 1 can be used to study eye coupled brain activities;

FIGS. 4A-4C show a few neurons/units that prefer a certain visual field location (in visual degrees), commonly referred to as “receptive-field” of a visual neuron in vision neuroscience, as obtained from an experimental system of FIG. 1 in a small primate study;

FIG. 5 shows preferred circuits for the FIG. 1 system for data collection and control and the general flow of data through the board; and

FIG. 6 shows a preferred virtual reality headset that includes a head mounted eye track system having eye tracking components of the FIG. 1 system.

### DETAILED DESCRIPTION OF THE PREFERRED EMBODIMENTS

Preferred embodiments include a headset-style camera system for animals larger than a rat (including humans) with a backpack-style data collection system. Two cameras, one for the world and one for finding eye position, are included in a head mount preferably configured to be stable and not easily displaced when worn by a freely moving animal Camera signals from the world and eye position cameras are sent to a microcontroller in the mounted system and synchronized. The complete system is wearable and includes components in a backpack, with no power tether.

Preferred systems output TTL (transistor-transistor logic) signals, which permit synchronization with many external systems, e.g., motion trackers, neural recording systems (EEG, Neural Spike recordings) and external camera systems. Preferred systems provide for detailed and complete synchronized data collection from the world and eye views. Preferred systems provided a powerful new tool for animal studies. Prior systems generally require animal restraint or at least don't allow true freedom of movement.

The present invention permits experiments in animals without restraining the animal Experiments with prototype of the invention have already revealed significant brain state changes when an animal is free, providing new insight into how the brain uses input from multiple sensory modalities while the animal is freely behaving.

An example prototype system uses STM32H7 microcontroller from STMicroelectronics to run 2 cameras. One camera has a macro lens attached to that is focused on the eye of the animal preferably through a hot mirror such that the animal field of direct forward view and a significant angle of peripheral view are unobstructed. The second camera (world cam) looks at in the same direction and general field of view of the animal. An IR LED is preferably used to illuminate the eye of the animal. The hot mirror reflects the IR into the eye and then the reflected light comes back to the eye camera. The eye camera also has preferably has a visible light filter which blocks wavelengths other than IR. This permits detection of the eye while excluding the visible light spectrum that was presented to the eye. Battery power is provided in the backpack, and the entire system is contained and untethered, allowing the animal to move both its head and body freely.

Preferred mountings are metal 3D printed with Titanium alloy that allows for sturdy and easily customizable parts. Preferred mountings are adjustable, to fit multiple animals of different sizes or even different types of animals. Preferably, the mounting is shaped according to a particular type of animal to comfortable mount.

A preferred system uses machine learning, e.g., a Deep Neural Network, to identify the pupil and superimpose the calibrated eye position on to the world camera images to know exactly where the animal was looking in the world. This allows tracking of the animals' gaze irrespective of the wide range of complex conjugated movement (head and eye combined) the animal is capable of making.

Preferred embodiments provide two cameras with a hot mirror (reflects IR but allows visible light to pass) disposed to capture eye movement from one of the cameras. Preferred systems use a microcontroller instead of a microprocessor to get more reliable timestamps of incoming frames. The microcontroller can provide formatted data as described above and can aid the synchronization. The mounting and overall design is very scalable, e.g. from animals larger than the size of a rat to significantly larger creatures including humans.

Preferred embodiments of the invention will now be discussed with respect to experiments and drawings. Broader aspects of the invention will be understood by artisans in view of the general knowledge in the art and the description of the experiments that follows.

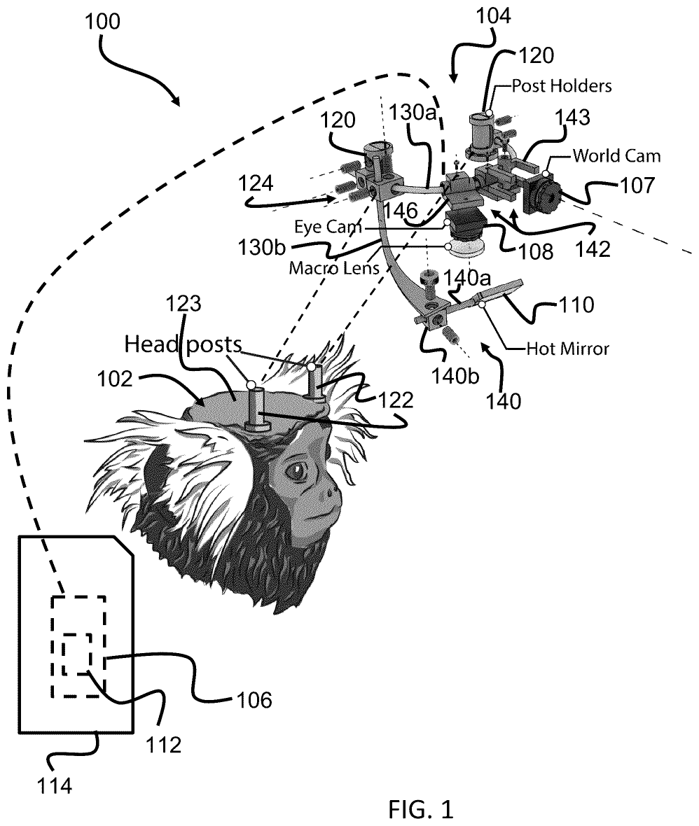

FIG. 1 shows a preferred head mounted camera and eye track system 100 for an animal A mount 102 is configured to the shape of the head of the animal, which can be accomplished, for example, using dental cement, Metabond®, or another adhesive during a minor surgery that fixes this mound/head-cap to the cranium of the animal A vision assembly 104 is configured to attach to the mounting. Data collection circuitry 106 communicates with the vision assembly 104, preferably via a flex cable.

The vision assembly 104 includes a forward-facing view camera 107 and an eye camera 108 with a macro lens and a visible light filter. A IR reflecting hot mirror 110 is arranged to be outside of a forward field of view of the animal while being angled to provide an image of an eye of the animal to the eye camera 108, while permitting the animal an unobstructed direct forward view and a substantial unobstructed peripheral view. The eye camera 108 preferably includes an illumination source, e.g., an IR LED, to illuminate the eye. The data collection circuitry 106 includes a microcontroller 112 receiving image data from the eye 107 and the view 106 cameras and is configured to synchronize the data such that the animal's gaze is synchronized with the image data. A backpack e.g. 114 of suitable material, e.g., leather, with power source (e.g., a 8 gm 400 mAh Li—Po battery) for the vision assembly 104 and the data collection circuitry 106 is configured to be worn by the animal. The mounting 102, vision assembly 104 and data collection circuitry 106 are untethered from external equipment such that the animal can move freely. Data collected is stored by the data collection circuitry 106 locally on an SD card or other suitable memory but can be wirelessly communicated for calibration purposes at lower frame rate. This approach ensures that the animal is never tethered during testing and can move about freely during any experiment.

The vision assembly 104 is held on the mounting 102 using small M4 set screws, such that it can be removed from the animal when not in use for the animal's comfort. The vision assembly 104 includes post holders 120 that lock onto posts 122 of the mounting 102, such as via setscrews or pins 124 or other mechanisms like spring loaded detents or clamps. Any suitable quick mount mechanism between posts and holders can be used. The posts 122 extend from a head plate 123 that is matched to a top of the animal's head and is surgically, adhesively or otherwise fixedly attached to the animal's head.

Curved main and side frame members 130a and 130b are fixed to the post holders 120. The frame members 130a and 130b can be separate or unitary, and mount the cameras 107, 108 and mirror 110. The frame members 130a and 130b, cameras 107, 108 and mirror are preferably lightweight and rigid. The forward frame member that mounts the cameras 107 and 108 is generally matched to a shape of the animal's forehead. The side frame member 130b curves away from the animal's forward field of view and preferably downward so that the mirror 110 is below the animal's field of view. A preferred material is titanium for its rigidity and lightness. Rigid plastics could also be used. The frame members 130a and 130b are preferably matched to curves of the animal's head.

A mirror mount 140 is preferably adjustable to permit adjustments that provide an optical path between an eye of the animal and the eye view camera 108. Camera mounts 142 are preferably adjustable by sliding along their respective curved frame member 130a to permit the eye camera 107 to provide the world view seen by the animal and the eye camera to see an eye of the animal via an optical path provided by the mirror 110.

The mirror mount 140 includes of two parts—a mirror rest 140a and a frame connector 140b. The thin hot mirror 110 can be fixed via adhesive to the hot mirror rest 140a. The mirror-rest is then held in the minor-rest-holder 140b via setscrews. This completed assembly is held by the side frame member 130b via setscrews that connects the hot mirror assembly to the main frame member 130a via a connector 124. This connector 124 and mirror rest holder allow the mirror to be moved up/down by moving the side frame member 130b and rotated using the mirror-rest-holder 140b to provide the best view of the eye.

A world cam mount 142 includes a base 143 for holding the world cam. The base includes a lumen to allow it to slide tightly onto the main frame member 130a. It base can have a hole tapped for an m2 screw and use a metal m2 screw to set the position of the holder 142 on the main frame member 130a. The base 143 includes 4 legs, which can be adhesively or otherwise attached to the world camera 107. An eye camera and LED holder(s) 146 are constructed similarly to the holder 142, but include a flat plate(s) for pointing the eye camera 108 and its integrated or separate LED downward.

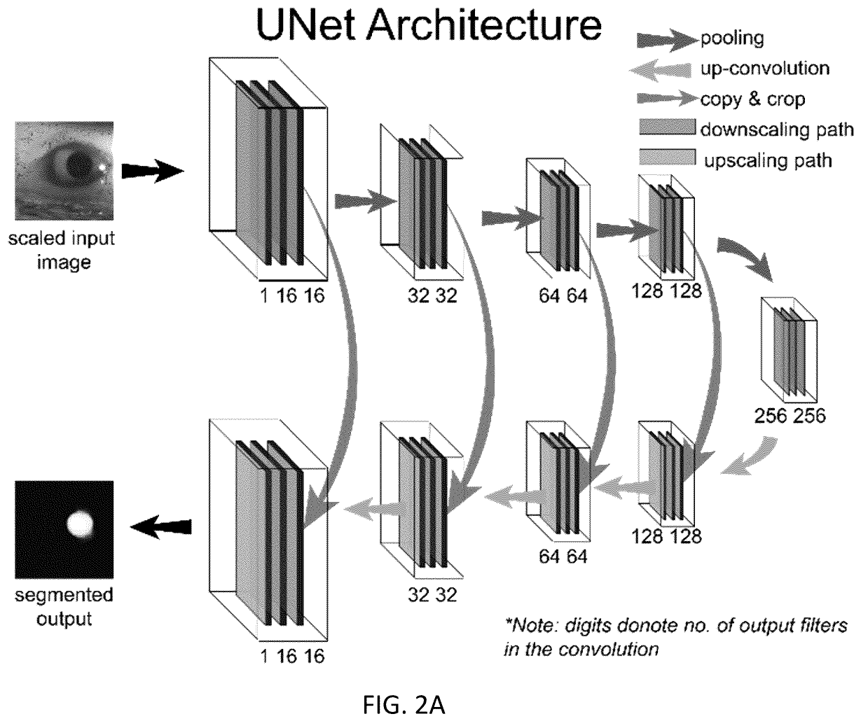

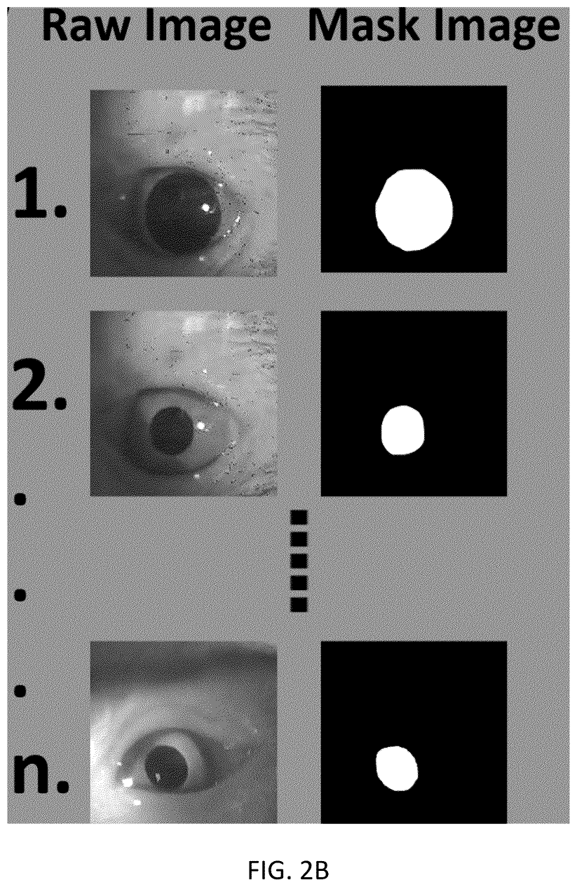

FIG. 2A shows a preferred UNET artificial neural network architecture used for pupil detection from recorded eye video. A conventional approach is a threshold-based approach for pupil detection. However, such an approach is likely to fail because there is no fine control of external lighting for a freely moving animal, and the threshold-based approach would likely fail. A preferred segmentation neural network shades the feature/object of interest as white while rest of the image is rendered black. The network is trained on paired dataset. Every pair has a raw image and a masked image as shown in FIG. 2B with iris of the eye colored white while rest of the image shaded black. These paired sets help the neural network to learn what to look for in an image (white portion of the mask) and what to ignore (in black). With this training, the network performs reliable pupil detection from images of the eye camera in varying light conditions. This is unlike methods that merely track a darkest spot of an eye, which is less reliable for gaze tracking. Instead, the UNET machine tracks eye features. The network is used in post-processing of the data once the experiment run has been completed.

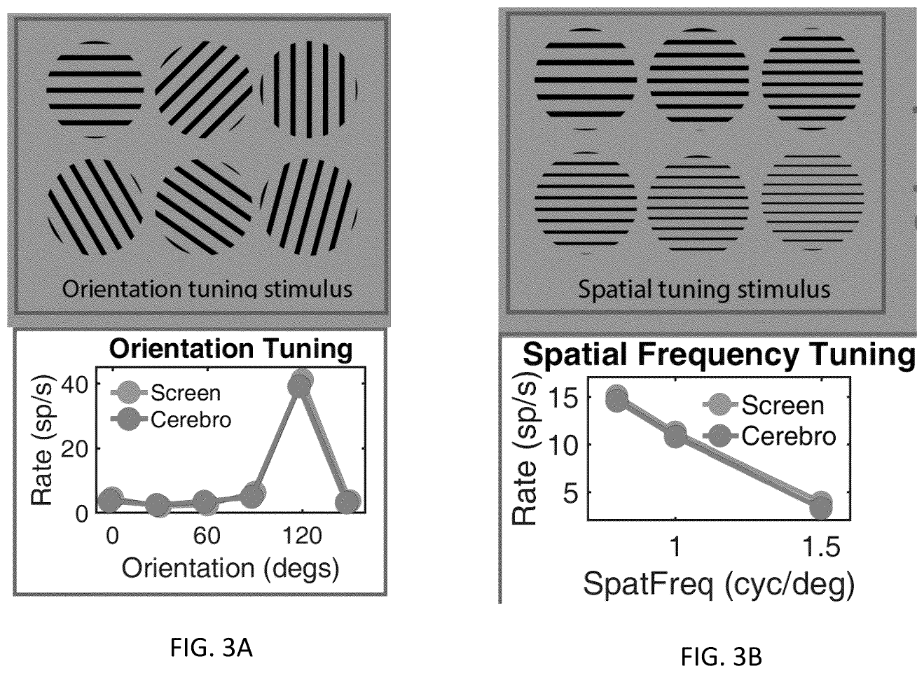

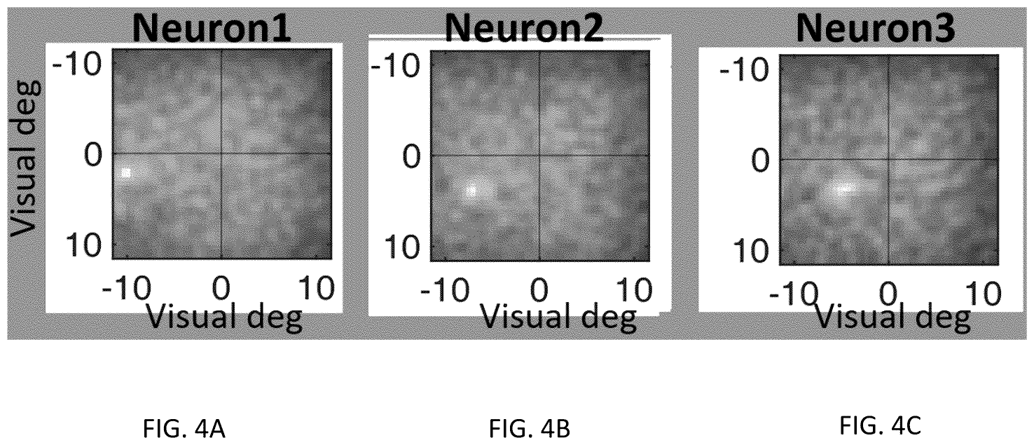

FIGS. 3A-3B shows that this eye-tracking system can be used to study eye coupled brain activities. To test this, the animal was shown gratings on the screen with different orientations (FIG. 3A) and spatial frequencies (FIG. 3B). FIG. 3A shows one example neuron that has a preference (shows more activity) for a certain orientation than the others. FIG. 3B shows an example neuron that has a preference (higher neural firing rate) for a certain spatial frequency of the stimulus. FIGS. 4A-4C show a few neurons/units that prefer a certain visual field location (in visual degrees), commonly referred to as “receptive-field” of a visual neuron in vision neuroscience. This, to our knowledge, is the first head mounted eye tracking system that shows receptive fields and tuning curves of primary visual neurons in a freely moving non-human primate.

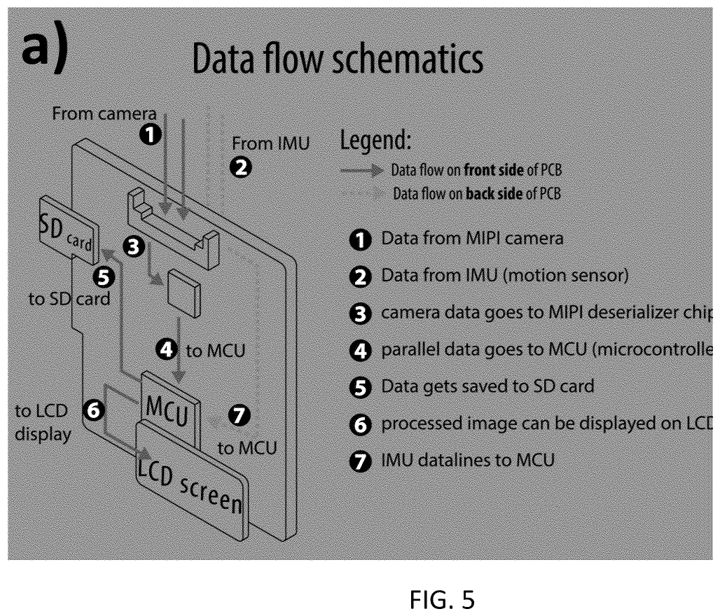

FIG. 5 shows preferred circuits for the PCB 112, and the general flow of data through the board. Two streams of data come to the microcontroller unit (MCU). First, there is camera data that gets serialized by a deserialized chip and then dropped on to the MCU for either display to the LCD screen or save to the SD card. Secondly, the motion data is collected using an IMU (Inertial Measurement Unit) and saved to the SD card via MCU. A PCB board used in experiments was small (40 mm×45 mm) and light weight (10 gm) and could be carried easily by a small non-human-primate such as common marmosets.

Details about a prototype consistent with the system 100 will now be discussed. 1. The mount as held to the animal's head using two 3D printed horns (posts) permanently glued to a plate shaped to mate with a top of the animal's head. The prototype consisted of parts printed with Titanium (Ti-6-Al-4V) using Direct Metal Laser Sintering (DLS) and are held together with a 2 mm (diameter) stainless steel rod curved to match the profile of the animal's forehead.

Headposts were glued to the headplate using acrylic. Covers were used on the headposts to protect them when the vision assembly was not being worn by the animal and to provide a smooth surface to minimize risk of the posts interfacing and fixing to external objects encountered by the animal.

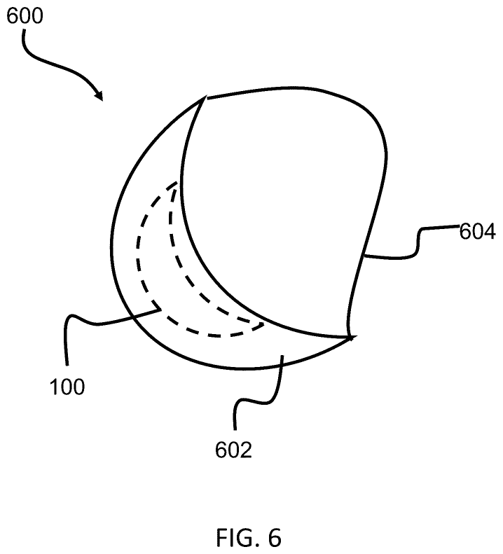

The experiments showed an example application to monitoring brain activity of small primates. Artisans will appreciate other applications. The mount can be configured, for example, to the shape of a human user. One additional application is to provide a better virtual reality experience by incorporating the invention into a virtual reality headset to monitor eye position (gaze direction) independently of head position. In virtual reality games, VR headsets track head movement/headset movement (with sensors on the headset). Tracking head movement alone can provide a poor simulation because a scene in front of a person in real life changes as a result of both head and eye movement. Tracking eye movement in addition can be leveraged to provide better view for the user. For example, if the VR system detects that both head and eye move in same direction, the scene on the headset is changed. However, if the head moves but the eye gaze remains fixed, the scene is not changed because the person is conducting a VOR (Vestibular-occular reflex) correction. FIG. 6 shows an example virtual reality headset 600 that includes a standard VR goggle 602 and strap 604 configured to be worn by a human user. The goggle 602 typically contains significant volume for video display to a human user, and that volume allows the camera and eye track system 100 to be incorporated into the google 602. Eye tracking is most important to this application, so the forward facing world camera 107 can be omitted, while the eye camera and eye tracking of the invention provide additional information for the VR system.

While preferred embodiments have been described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. A head mounted camera and eye track system for an animal, comprising:
a mount configured to the shape of the head of the animal, a vision assembly configured to attach to the mounting, and data collection circuitry;
wherein the vision assembly comprises a forward-facing view camera, an eye camera, and a mirror arranged to be outside of a forward field of view of the animal while being angled to provide an image of an eye of the animal to the eye camera;
wherein the data collection circuitry comprises a microcontroller receiving image data from the eye and the view cameras and configured to synchronize the data such that the animal's gaze is synchronized with the data; and
a power supply for the vision assembly and the data collection circuitry, the power supply being configured to be worn by the animal, and
wherein the mounting, vision assembly and data collection circuitry are untethered such that the animal can move freely.

2. The system of claim 1, wherein the microcontroller synchronizes animal gaze data and world view data.

3. The system of claim 1, further comprising an illumination source that illuminates the animal's eye.

4. The system of claim 3, wherein the illumination source comprises an IR LED.

5. The system of claim 4, wherein the mirror comprises a hot mirror that reflects IR light and passes visible light.

6. The system of claim 5, wherein the eye camera comprises a visible light filter that passes IR light.

7. The system of claim 1, comprising machine learning trained to identify the pupil and superimpose a calibrated eye position on to the world camera images.

8. The system of claim 1, wherein the machine learning is trained on a paired dataset or pairs of raw eye image and a masked images in which the iris of the eye is colored white while rest of the image shaded black.

9. The system of claim 1, wherein the vision assembly comprises a main curved frame matching a curve of the animal's head.

10. The system of claim 9, wherein the forward-facing view camera and the eye camera are mounted on the main curved frame.

11. The system of claim 9, wherein the main curved frame comprise post holders configured to lock onto posts of the mount.

12. The system of claim 11, wherein the posts extend from a head plate that is matched to a top of the animal's head and is configured to be surgically or otherwise fixedly attached to the animal's head.

13. The system of claim 12, comprising a side curved member that mounts the mirror, the side curved member being configured to curve away from the animal's forward field of downward so that the mirror is below the animal's field of view.

14. The system of claim 13, wherein the mirror comprises an adjustable mirror mount that permits adjustments that provide an optical path between an eye of the animal and the eye view camera.

15. The system of claim 1, wherein the mount is configured to be surgically or otherwise fixedly attached to the animal's head and the vision assembly is configured to be removably attachable to the mount.

16. The system of claim 15, wherein the mount comprises titanium.

17. The system of claim 16, wherein the mount comprises posts that extend from a head plate that is matched to a top of the animal's head and is configured to be surgically or otherwise fixedly attached to the animal's head.

18. The system of claim 1, wherein the mount and vision assembly are part of a virtual reality headset.

19. The system of claim 1, comprising a backpack configured to be worn by the animal, the backpack containing the power supply.

20. A head mounted eye track system for an animal, comprising:
a mount configured to the shape of the head of the animal, a vision assembly configured to attach to the mounting, and data collection circuitry;
wherein the vision assembly comprises an eye camera, a mirror arranged to be outside of a forward field of view of the animal while being angled to provide an image of an eye of the animal to the eye camera, and an illumination source that illuminates the animal's eye;
wherein the data collection circuitry comprises a microcontroller receiving image data from the eye camera and configured to provide synchronization information regarding the image date from the eye camera; and
a power supply for the vision assembly and the data collection circuitry, the power supply being configured to be worn by the animal, and
wherein the mounting, vision assembly and data collection circuitry are untethered such that the animal can move freely.
